(19) United States
(12) Patent Application Publication
(10) Pub. No.: US 2008/0269879 A1
(43) Pub. Date: Oct. 30, 2008

(54) Implantable Prosthetic Vascular Valve

(76) Inventors: Rahul Dilip Sathe, San Clemente, CA (US); David N. Ku, Decatur, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/996,791

(22) PCT Filed: Jul. 26, 2006
(86) PCT No.: PCT/US06/28858

§ 371 Date: Jul. 7, 2008

Related U.S. Application Data
(60) Provisional application No. 60/702,971, filed on Jul. 27, 2005.

(51) Int. Cl. A61F 2/24

(52) U.S. Cl. 623/2.12

(57) ABSTRACT

The present disclosure generally relates to implantable, prosthetic vascular valves, methods of making the valves, and methods of using the valves, in particular, in the human venous system.

### CROSS-REFERENCE TO RELATED APPLICATIONS

This application claims priority to copending U.S. provisional patent application Ser. No. 60/702,971, entitled “Implantable Prosthetic Vein Valve” filed on Jul. 27, 2005, which is entirely incorporated herein by reference.

### FIELD OF THE INVENTION(S)

This present disclosure is directed to an implantable prosthetic venous valve designed to replace a diseased, damaged, or clinically incompetent valve in the human venous system. It is recommended for, but not limited to, implantation in the deep veins of the lower extremities in humans.

### BACKGROUND

The human venous system in the lower extremities contains a number of one-way valves that function in allowing forward (antegrade) blood flow to the right atrium of the heart while preventing reverse (retrograde) flow to the feet. Using the muscle action of the calf, or the “peripheral heart,” the body is able to overcome gravitational forces to maintain blood flow back to the heart. The valves thus prevent blood from pooling in the lower extremities. Physiologically functioning valves are capable of withstanding very high proximal pressure gradients with minimal leakage, and can open at very low distal pressure gradients. However, for many patients, venous function is severely compromised by chronic venous disease (CVD), caused by chronic venous insufficiency (CVI).

CVI affects nearly one million new patients every year, and causes health problems such as varicose veins, ulceration, swelling, and in the more severe cases deep vein thrombosis and pulmonary embolism. Venous reflux causes 80 to 90 percent of CVI and is the result of incompetent venous valves. The most common type of incompetence, secondary incompetence, often results in complete destruction of the valve leaflets. Venous reflux due to secondary incompetence is rarely surgically repaired, and when it is, the repair seldom lasts. When secondary incompetence occurs in the deep venous system, valve replacement is the only viable treatment.

There are two main options in deep venous valve replacement: 1) transplantation or transposition and 2) prosthetic implantation. The first vein valve autotransplant in a human patient in was performed in 1982.

However, even after more than 20 years of refinement, the surgery is still used only in necessary cases, only after medication, physical rest and therapy, and other less invasive surgical procedures have been tried or considered. Valve transplant or transposition can cause unnecessary trauma to the patient's leg, and most procedures require indefinite post-operative anti-coagulation treatment. Problems may also arise even prior to surgery; for instance, it can be difficult to find a suitable donor valve. This is evidenced by the fact that 30 to 40 percent of auxiliary vein valves, which are often used for superficial femoral venous valve replacement, are found incompetent prior to harvesting. The challenges of using a native vein valve for transplantation or transposition thus increase the need for a suitable prosthetic vein valve.

There has yet to be a prosthetic venous valve developed that has demonstrated the necessary functional performance for operating satisfactorily in human physiologic conditions. While various designs have been pursued in the past, many such designs possess shortcomings that prevent them from being a sufficiently functional design.

In more recent years, a variety of mechanical and bioprosthetic implantable valves have been created and studied; however, very few have shown to be suitable for human implantation. Attempts have been made at using valves have been fabricated of gluteraldehyde-fixed umbilical cord segments, but implantation studies proved ultimately unsuccessful, due to blockages and other problems. Attempts have also been made at using bioprosthetic gluteraldehyde-fixed cardiac valves as vein valves. However, gluteraldehyde fixation creates valves that are typically too stiff for the venous system; also, cardiac valves are not properly designed for the venous system. Attempts at using cryopreserved human vein valves have also been made. However, it has been reported that cryopreserved venous valve allografts resulted in occlusion, morbidity and generally poor results. Additionally, the majority of cryopreserved valves require additional repair prior to implantation.

Various prosthetic attempts have concentrated on designing mechanical venous valves, but in vitro and in vivo (canine) experiments, have ultimately proved unsuccessful. Some prosthetic valves incorporate a rigid stent or support structure. Some such previous designs describe valves for cardiac use in which, when no external forces are applied, the leaflets are separated from contacting each other. Such a device is more appropriately designed for the high flow, velocity-sensitive cardiac valve system, not for the low flow, pressure-sensitive venous valve system. The leaflets of such a design may leak, and the valve's rigid commissural support system may impose damage to the vein wall.

The literature also describes the fixation process of chemically treating autologous or heterologous bio-prosthetic venous valve vein segments. Fixation of such biological valves usually involves gluteraldehyde, an agent that crosslinks collagen, creating a stiffer valve and valve leaflets. Stiff valve leaflets may not open at the low physiological pressure gradients of the venous system, and could block blood flow.

Other existing prosthetic valve designs also have shortcomings, such as valves that do not properly seal against reflux or under high pressures, valves with complicated leaflet designs that would be difficult to manufacture, valves that impose significant radial force on the vein, leading to local stress concentrations, defects and trauma, and valves that are so resistant to antegrade flow that they may not open properly under normal physiologic pressure gradients, thereby blocking blood flow. Thus, many of the above-discussed techniques have shortcomings and disadvantages, and there is a need in the industry for an implantable prosthetic vein valve that overcomes at least some of these shortcomings and disadvantages.

### SUMMARY

The present disclosure generally relates to prosthetic valves and methods of use and manufacture thereof. The valve of the present disclosure can be used in non-biological systems, but is generally designed as an implantable, prosthetic valve for use in the human vascular system, particularly the venous system. The valve is particularly suited for use in the venous system because it allows antegrade blood flow towards the heart when subjected to a very low distal pressure gradient (e.g. that caused by contraction of leg muscles), while also preventing retrograde blood flow and leakage when subjected to physiologically high proximal pressure. In various embodiments the valve is also biocompatible, flexible, has low thrombogenicity, and is sufficiently durable to withstand multiple cycles of opening and closing in physiologic conditions.

Described briefly, in an embodiment of the present disclosure, among others, the valve is made of a generally cylindrical tube having a central portion, an inlet, an outlet, and at least two leaflets. The leaflets are in the closed position (e.g. contacting one another) in a relaxed state. The leaflets open upon the application of pressure from the direction of the inlet (e.g. a distal pressure gradient) to form an orifice and allow a fluid to flow through the orifice to the outlet. Since the leaflets are in the closed position in the relaxed state, this substantially prevents backflow/reflux of fluid back through the valve from the outlet to the inlet. In preferred embodiments, the valve has a flared outlet and flared inlet to allow the valve to more closely approximate the geometry of the vein when in its distended state. In some embodiments, the valve is made of a flexible material to further enhance the performance of the valve. The valve may be designed to have varying flexibility in the different valve components. For instance, in some embodiments the compliance of the inlet, outlet, and leaflets is greater than that of the central portion of the tube.

The valve of the present disclosure can be implanted in a patient by standard procedures, including, but not limited to, a minimally invasive catheter procedure or more conventional surgical procedures (e.g. a venotomy), and can be affixed to its position via various methods known to those in the art including, but not limited to, sutures, a stent or stent system, and hooked or barbed protrusions.

Other aspects, methods, devices, features, and advantages of the present disclosure will be or become apparent to one with skill in the art upon examination of the following drawings and detailed description. It is intended that all such additional systems, devices, methods, features, and advantages be included within this description, be within the scope of the present disclosure, and be protected by the accompanying claims.

### BRIEF DESCRIPTION OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B are isometric views of an embodiment of the valve prosthesis according to the present disclosure in the closed configuration (FIG. 1A) and the open configuration (FIG. 1B).

FIGS. 2A and 2B are cross-sectional side views of an embodiment of the valve prosthesis according to the present disclosure in the closed configuration (FIG. 2A) and the open configuration (FIG. 2B).

FIGS. 3A and 3B are cross-sectional top views of an embodiment of the valve prosthesis according to the present disclosure in the closed configuration (FIG. 3A) and the open configuration (FIG. 3B).

FIG. 4 is a side view of an embodiment of the valve prosthesis according to the present disclosure.

FIG. 5 is an isometric view of an embodiment of the valve prosthesis according to the present disclosure.

FIG. 6 is an end view (proximal) of the outlet of an embodiment of the valve prosthesis according to the present disclosure in the closed configuration.

FIG. 7 is an end view (distal) of the inlet of an embodiment of the valve prosthesis according to the present disclosure in the closed configuration.

FIG. 8 is a graph illustrating the opening pressure of an embodiment of the valve of the present disclosure after various numbers of cycles of opening and closing, and showing that the valve of the present disclosure consistently meets the design criteria after at least 500,000 cycles of opening and closing.

FIG. 9 is a graph illustrating the leakage rate of an embodiment of the valve of the present disclosure after various numbers of cycles. This figure illustrates that the valve allows minimal leakage when exposed to backpressure, and performance is comparable when the valve is new as well as after opening and closing after 500,000 cycles.

### DETAILED DESCRIPTION

For a prosthetic implantable vein valve to be optimally functional, the valve should typically have the following features. The valve should be able to open and allow antegrade (forward) flow with little resistance. The valve should also withstand physiologic proximal pressures of about 100 mm Hg or greater, while preventing reflux (reverse flow) and keeping leakage less than about 1.0 mL/minute. The valve should have low thrombogenicity, should cause minimal pain to the patient, and should have the durability to last at least about 500,000 cycles. The valve should not become obstructed after implantation, lest it block blood flow. Prosthetic valves should also preferably meet the demands of vein dispensability, as vein diameter expands from about 1.4 to about 2.0 times the normal vein diameter when subjected to pressures of only about 50 mm Hg. Ideally, this means the prosthetic valve should flexibly conform to the curves and bends of veins.

In general, the present disclosure provides an implantable prosthetic valve designed to meet the functional criteria, set forth above, of a valve placed in the venous system. It is designed to allow substantially unrestricted antegrade flow (e.g., allows antegrade flow under a pressure gradient of about 5 mmHg or less), minimize reflux and leakage, and is also easy to manufacture. It is designed to be biocompatible, have low thrombogenicity, and can also be used in other body vessels, particularly those that conduct primarily unidirectional flow. It can also be used in non-biological systems.

The valve of the present disclosure is designed to accommodate the anatomy and mechanical properties of veins. It is also designed to be implantable via several methods, including, but not limited to, intravenous stent delivery or transluminal suturing techniques. Those of skill in the art will appreciate that other delivery and attachment methods may be developed and could be practiced with embodiments of the prosthetic valves of the present disclosure.

Valve prototypes according to embodiments of the present disclosure, described in greater detail below, were manufactured and bench tested. Such testing demonstrated the operational functionality of the valve. Three critical design criteria were defined for evaluating the valve's functional performance. The criteria included that the valve be able to do the following: 1) withstand at least about 300 mmHg of backpressure with less than about 1.0 mL leakage per minute, 2) open with a pressure gradient less than about 5 mmHg, and 3) meet criteria 1 and 2 even after about 500,000 cycles of opening and closing in simulated physiologic conditions.

As described in greater detail in the Examples below, bench testing of an embodiment of the valve of the present disclosure demonstrated that the valve met these three critical design elements. The valve consistently opened with pressure gradients as low as about 2.0±0.5 mmHg, and was able to withstand up to about 300 mmHg of physiologic proximal pressures with a leak-rate less than about 0.3 mL/min. The valve remained functional even after opening and closing over 500,000 times. The burst pressure of the valve was about 530±10 mmHg, six times greater than physiologic pressure in leg veins. These results indicate that the valve is operationally functional and is a good potential solution to treating deep valvular incompetence in CVI patients. Additional detail about the tested valve embodiment can be found in the Examples below.

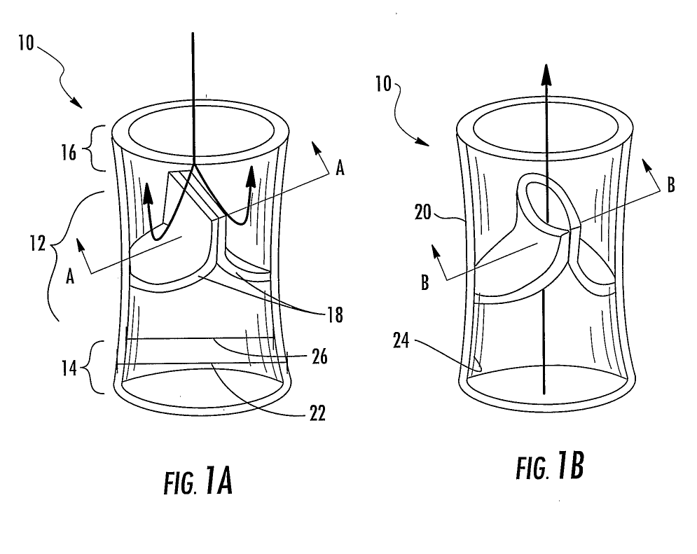

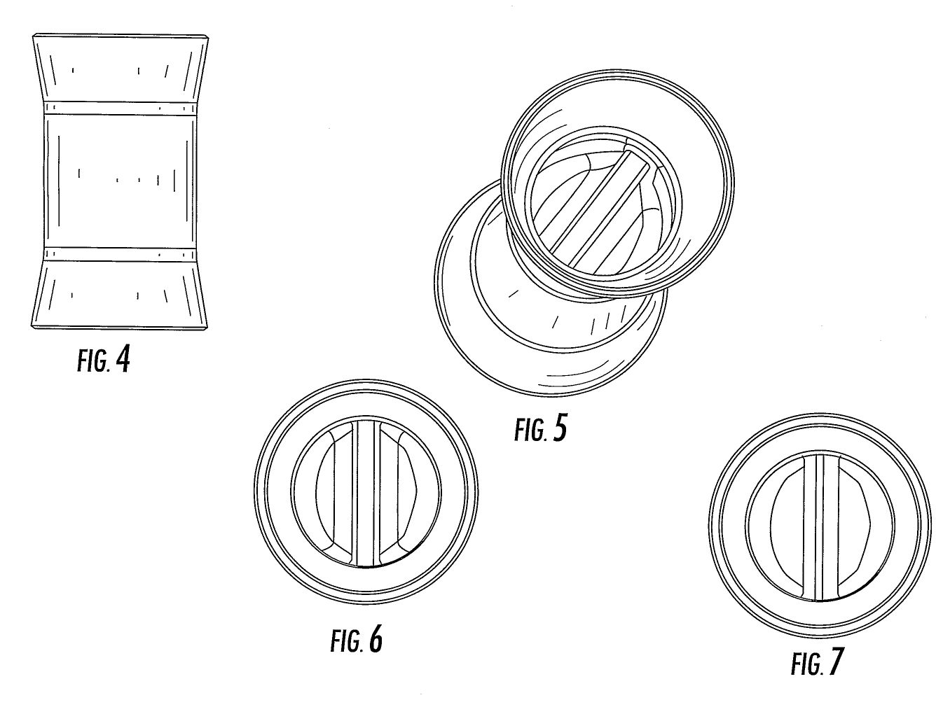

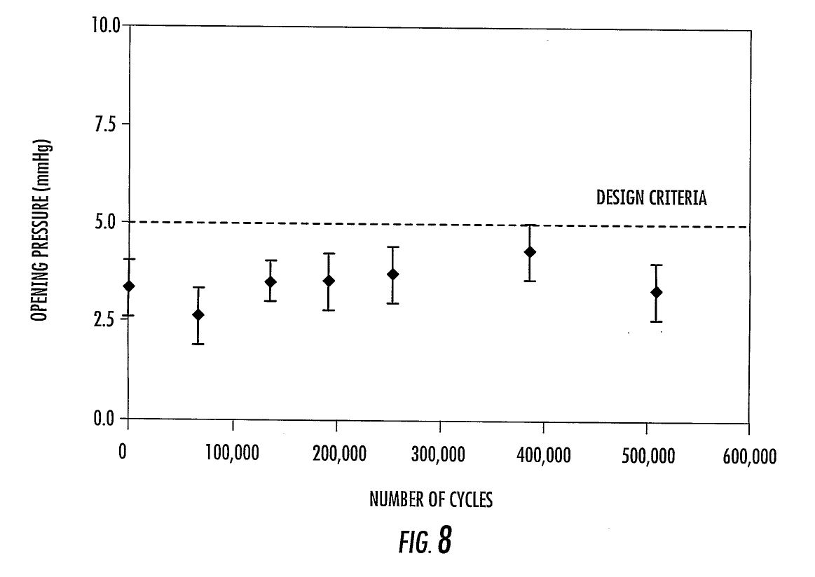

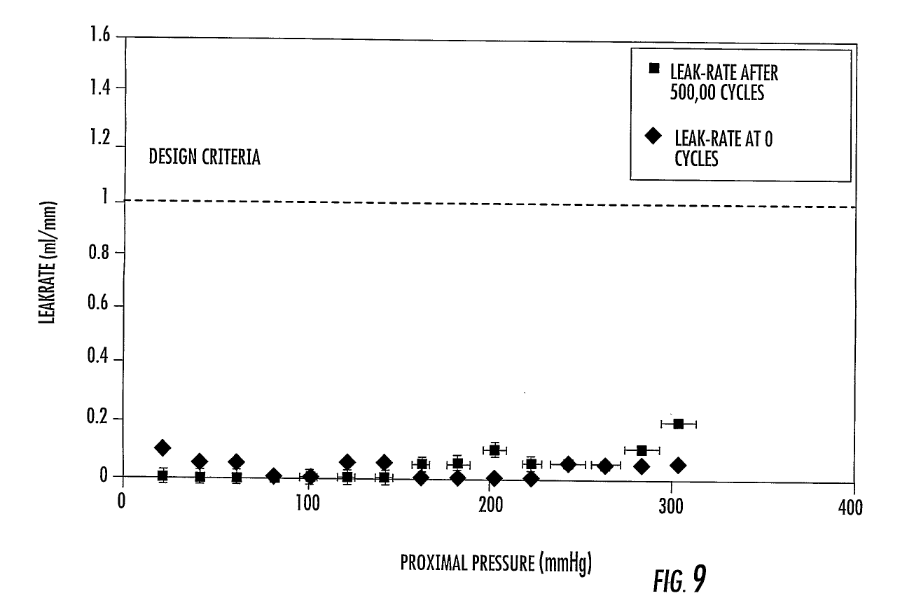

FIGS. 1-7 depict various views of an embodiment of a valve of the present disclosure and its various components. Referring now to the representative embodiments illustrated in FIGS. 1-3, the valve of the present disclosure includes a flexible, biocompatible, generally cylindrical tube 10 with a central portion 12, an inlet 14, an outlet 16, and leaflets 18. As used herein “generally cylindrical” refers to a valve tube having a shape that approximates a cylindrical form, but that is not necessarily perfectly cylindrical (e.g., the diameter may not be constant throughout the entirety of the “cylindrical” tube). The tube 10 also has an outer surface 20, defining an outer diameter 22, and an inner surface 24, defining an inner diameter 26. In preferred embodiments of the valve, the inlet 14 and outlet 16 are flared. The concept of a flared inlet and/or outlet is multi-purposeful. It facilitates circumferential sealing of the valve against the intimal layer of the vein wall. Circumferential sealing substantially prevents leakage during retrograde flow (e.g., allows a leak rate of less than about 0.3 mL/min under backpressures of about 300 mmHg or greater), and the flared inlet and outlet ensure that a good seal is created. The flared inlet and outlet also help alleviate stress concentrations in the vein wall during distension. In essence, the inlet and outlet are designed to help smoothly transition the vein wall from a diameter equivalent to the vein valve to the vein's distended diameter, minimizing damage to the vessel wall.

In embodiments, the valve also has the ability to expand elastically, in the radial direction, axial direction, or both. The entire valve may be elastically expandable, or certain portions of the valve may be elastically expandable to various degrees. For instance, in embodiments, the valve can elastically expand in the radial direction in the central portion of the tube and increase its radius by a value of about 0 R to about 0.5 R, preferably by at least about 0.2 R, where R is the inner radius of the central portion of the tube. During such expansion, the valve does not tear or break and experiences negligible plastic deformation.

Embodiments of the valve can elastically expand in the radial direction at the flared inlet and/or outlet, increasing in radius by a value of about 0 R to about 1.0 R, preferably by about 0.5 R, where R is the inner radius of the central portion of the tube. During such expansion, the valve does not tear or break and experiences negligible plastic deformation.

During expansion of the outlet, the valve's leaflets allow less than about 0.3 mL/min of leakage against an applied proximal pressure of at least about 300 mmHg, even when the flared outlet has increased in radius by at least about 0.5 R. This is a particularly advantageous attribute of the valve's design because various physiological venous conditions subject veins and vein valves to such elastic expansion. The ability of a valve according to the present disclosure to elastically expand at its outlet to match the dilation of a vein while the size and shape of the central portion of the valve tube and the inlet remain essentially unaffected ensures functional sealing of the valve against the vein wall even under the highest physiologic proximal pressures.

In embodiments, the valve can also elastically expand in length by a value of about 0L to about 0.5L, preferably by at least about 0.3L, while experiencing negligible plastic deformation, without tearing or breaking, where L is the total length of the valve in the axial direction.

The tube 10 of the valve includes two leaflets 18, which are preferably free of any framework or filaments, meeting each other in surface area contact to create a robust sealing mechanism. When the valve is in its relaxed state, meaning that it is free from any applied external forces, the leaflets 18 contact each other without exerting force on each other. This absence of a spring bias in the leaflets ensures that resistance to antegrade flow is minimized, a desirable attribute of a valve design, considering that it is a low physiologic pressure gradient that drives venous blood flow. Also, the valve, being closed in its relaxed state, helps control reflux of blood during retrograde flow better than a valve that is open in its relaxed state.

In some preferred embodiments of the valve of the present disclosure, the central portion 12 has a greater elastic modulus, and hence less compliance, than the leaflets 18 and the flared inlet 14 and flared outlet 16. This allows the leaflets 18 and inlet 14 and outlet 16 to better conform to tortuous venous anatomy and vein dilation, while the less compliant central portion of the tube 12 maintains the structural integrity of the valve at high proximal pressure. Leaflets that are fairly compliant help ensure proper sealing, and reduce the resistance of flow during valve opening.

The valve of the present disclosure can be fabricated using a single material that is cast or injected into a mold. This makes the production of the valve of the present disclosure fairly simple and economic, and the benefits of the financial and temporal savings can be passed along to the patient and surgeon. Preferably, the material used to make the valve is biocompatible and has low thrombogenicity. Suitable materials include, but are not limited to, polyurethanes, polyesters, polyethylenes, hydrogels, silastics, collagens, elastins, Room Temperature Vulcanized (RTV) rubbers, and silicones. A second material in a particulate form such as, but not limited to, fibers, filaments, and/or grains can be added into the valve tube's dominant material to create a composite material, altering the stiffness and improving the fatigue life of the valve. Such alterations can be made to the entire valve or only to certain portions of the valve (e.g., the central portion 12). This aspect of the design is advantageous in that it gives the manufacturer and the surgeon the ability to tailor valve of the present disclosure to a patient's specific clinical needs.

The valve of the present disclosure does not require a rigid frame, scaffold, or support structure. This is advantageous for at least two reasons. Primarily, it allows the valve to match the natural contours of the vein during most any physiological state, thus minimizing damage to the vein wall. Secondarily, it allows the valve to be collapsed into a catheter delivery system for minimally invasive implantation techniques.

The valve of the present disclosure can be implanted into a patient through several modes known to those of skill in the art. To minimize trauma, pain, and potential for infection, the valve can be delivered to the implantation site via an intravenous catheter. The flexibility and durability of the valve make it highly deliverable via a catheter. The valve can then be fixed into position using a fixation device, such as, but not limited to, a balloon-expandable stent, a self-expanding stent, hooks or barbs, or other endovascular implantation techniques known to those in the field.

An exemplary mode of implantation and fixation involves first delivering valve of the present disclosure to the implantation site, and then securing it inside the vessel using sutures or other suitable fixation techniques known to those of skill in the art. Delivery can be accomplished by performing a venotomy, which involves making a longitudinal incision through the wall of the vessel. The incision should be long enough to stretch open the vein wall and insert the valve by hand. Delivery can also be accomplished via an intravenous catheter, thus avoiding the need to cut through the wall of the vessel. After delivery, the valve is preferably fixed in its position using sutures. Generally, non-absorbable sutures are used for venous surgery, and are comprised of materials such as, but not limited to, silk or polypropylene. In particular regard to vascular surgery, suture size preferably ranges from about 5-0 to about 8-0. Interrupted sutures will give the greatest knot security, and can be placed in a longitudinal or circumferential direction, through the inlet and outlet of the valve. The number of sutures needed per valve will vary due to the diameter of the valve.

When using any mode of delivery, preferably the valve is positioned such that the plane in which the leaflets meet in surface contact is tangent to the circumferential direction of the limb. This is allows the valve to perform appropriately even when compressed by the deep fascial muscular pressure.

It may be advantageous to facilitate intimal growth and healing of the vessel to improve circumferential sealing of the valve. This can be accomplished by incorporating a woven, knitted, or otherwise porous sheath of biocompatible material onto the outer diameter of the valve tube and optionally the flared inlet and outlet. Suitable materials include, but are not limited to, polyethylene terephthalate (PET), expanded polytetrafluoroethylene (ePTFE), or a similar material that has been shown to facilitate intimal growth in vascular graft applications.

In certain embodiments of the present disclosure, anti-thrombogenic or thrombolytic agents including, but not limited to, heparin, sodium warfarin, or albumin are incorporated with the valve to help improve the response of the surrounding tissue and fluid to the introduction of a prosthetic valve. In such embodiments the agents may be incorporated on the surface of the valve of and/or into the valve material, and can be released actively or passively, at varying rates.

In yet other embodiments of the present disclosure, a radiopaque material is incorporated with the valve to allow a clinician to track the motion and position of the valve during catheter delivery via fluoroscopy. This is advantageous because the valve's performance will be optimized if placed in the correct location, and this method allows the clinician to accurately know the valve's location inside the body at any given time during the implantation procedure.

Embodiments of the present disclosure entail designing the valve based on venous anatomy, physiology, and local biomechanics. Embodiments also entail fabricating the valve in a manner that is economical, timely, tailored to allow appropriate quality control measures, makes use of readily available materials, and allows customizing the design for specific clinical needs.

The shape and size of the valve of the present disclosure impact the efficacy of the valve. Preferably, the valve is sized relative to the vessel that it will be implanted in. When implanting the valve of the present disclosure into human deep veins, the outer diameter (e.g., 22 in FIG. 1A) of the cylindrical tube preferably ranges from about 0.75 D to about 1.50 D, where D is the un-collapsed inner diameter of the vein at low pressures. More preferably, the tube outer diameter 22 is from about 0.9 D to about 1.3 D, and most preferably, from about 1.0 D to about 1.2 D. In certain embodiments the outer diameter of the central portion of the cylindrical tube is from about 1 millimeter to about 50 millimeters.

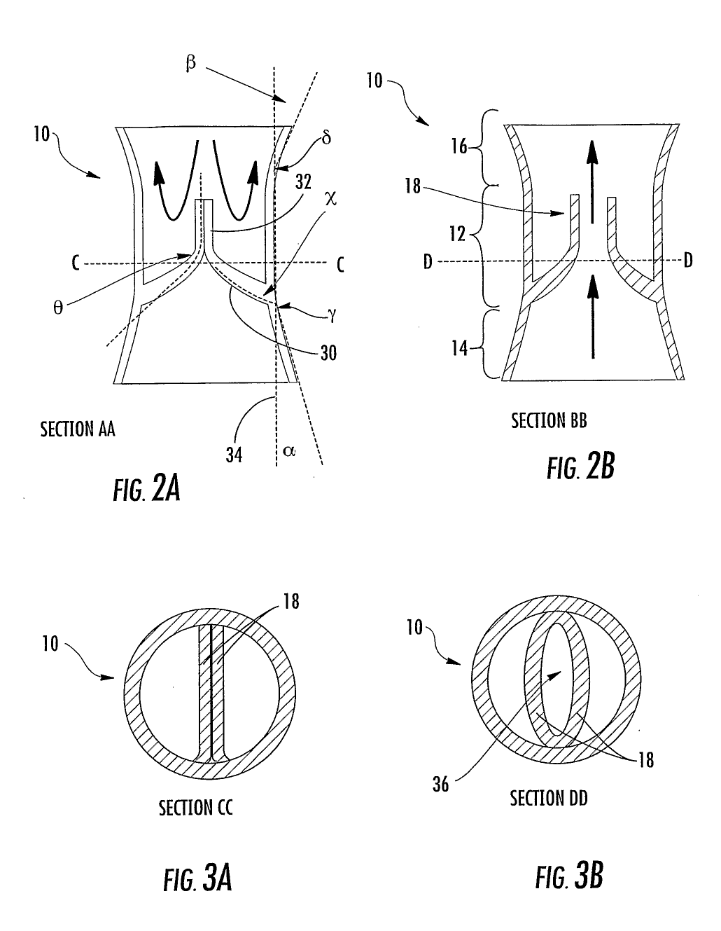

As shown in FIG. 2, the angle α formed between the flared inlet 14 and a line 34 running substantially parallel to the outer surface 20 of the central portion 12 of the tube (and tangent to the outer diameter 22 of the central portion 12 of the tube), is preferably about 0 degrees to about 50 degrees, more preferably about 0 degrees to about 35 degrees, and most preferably about 0 degrees to about 20 degrees. (As used herein, “substantially parallel” indicates that one object or line may be exactly parallel to or is so nearly parallel to another object or line that it appears to be parallel to the other object or line.) An angle γ, supplementary to angle α, is formed by the intersection of a line running substantially parallel to the outer surface of the wall of the flared inlet 14 and a line 34 running substantially parallel to the outer surface of the central portion of the tube 12. Angle γ is preferably about 90 degrees to about 270 degrees, more preferably about 120 degrees to about 180 degrees, most preferably about 155 degrees to about 180 degrees. Preferably, the joint formed by angle γ (e.g., the joint formed by the meeting of the flared inlet and the outer surface of the central portion of the generally cylindrical tube) is smoothed with a variable-radius fillet in such a manner that a parametric surface is created that can be described with radii of about 0 R to about 10 R, more preferably of about 2 R to about 8 R, and most preferably of about 3 R to about 7 R, where R is the inner radius of the central portion of the tube.

Similarly, the angle β formed between the flared outlet 16 and a line 34 running substantially parallel to the outer surface 20 of the central portion 12 of the tube (and tangent to the outer diameter 22 of the central portion 12 of the tube) preferably is about 0 degrees to about 50 degrees, more preferably about 0 degrees to about 35 degrees, and most preferably about 0 degrees to about 20 degrees. Angle δ, supplementary to angle β, is formed by the intersection of a line running substantially parallel to the outer surface of the wall of the flared outlet 16 and a line 34 running substantially parallel to the outer surface of the central portion of the tube 12. Angle δ is preferably about 90 degrees to about 270 degrees, more preferably about 120 degrees to about 180 degrees, and most preferably about 155 degrees to about 180 degrees. The joint formed by angle δ (e.g., the joint formed by the meeting of the flared outlet and the outer surface of the central portion of the generally cylindrical tube) is also preferably smoothed with a variable-radius fillet in such a manner that a parametric surface can be described with radii ranging from about 0 R to about 10 R, more preferably ranging from about 2 R to about 8 R, and most preferably from about 3 R to about 7 R, where R is the inner radius of the tube.

The length of the entire valve is preferably about 0.5 D to about 4 D, more preferably about 1 D to about 4 D, and most preferably about 2 D to about 3 D. The length of the flared inlet and outlet is preferably about 0.2 D to about 1 D, and most preferably about 0.4 D to about 0.8 D. In certain embodiments, the length of the valve is about 2 millimeters to about 50 millimeters. The thickness of the valve wall (e.g., the wall of the tube portion of the valve, having an inner and outer surface as defined above) is preferably about 0.01 D to about 0.2 D, and most preferably about 0.05 D to about 0.15 D.

The thickness of the leaflets is preferably about 0.01 D to about 0.2 D, and most preferably about 0.05 D to about 0.15 D. Preferably, the valve has two or more leaflets. Most preferably, there are two leaflets. This will keep the valve relatively simple to manufacture, and will make the valve more robust.

The leaflet shape in embodiments of the valve of the present device is unique. In a preferred embodiment, each leaflet is neither a parabolic shape, nor quite an elliptical shape, but rather is a combination of an ellipsoidal shape and approximately trapezoidal plate. The shape of the designed leaflets minimizes the fluid drag and resistance during the opening process in antegrade flow, yet improves the sealing ability during closure in retrograde flow.

In embodiments of a valve containing two leaflets 18, as shown in FIG. 2, each leaflet's distal half 30 (e.g., the portion of the leaflet more distally positioned than the other portion of the leaflet) forms an angle χ with a line 34 running substantially parallel to the outer surface 20 of the central portion 12 of the tube (and tangent to the outer diameter 22 of the central portion 12 of the tube). Angle χ is preferably about 15 degrees to about 75 degrees, more preferably, it is about 30 degrees to about 60 degrees. Angle χ is thus formed by the joining of the distal half 30 of each leaflet with the central portion 12 of the tube. The joint that forms angle χ is then preferentially smoothed with a variable-radius fillet in such a manner that a parametric surface is created having curvatures of various radii ranging from about 0.5 D to about 10 D.

In preferred embodiments, the leaflet's distal portion 30 has a shape that approximates half of an elliptical plate. Also, in some preferred embodiments, the leaflet's proximal half 32 (e.g., the portion of the leaflet more proximally positioned than the other portion of the leaflet) approximates a trapezoidal plate orientated so that it is substantially parallel to a plane containing the longitudinal axis of the valve. The distal and proximal halves 30 and 32, respectively, of each leaflet 18 connect to form an angle θ, which is preferably about 100 degrees to about 170 degrees, more preferably about 125 degrees to about 155 degrees. Angle θ is thus formed by the joining of the distal half 30 of each leaflet with the more proximal half 32. In some embodiments, the joint that forms angle θ is preferentially smoothed with a variable-radius fillet in such a manner that a parametric surface is created having curvatures of various radii ranging from about 0.5 D to about 5 D.

The valve of the present disclosure is preferably biocompatible, non-thrombogenic, and non-immunogenic. In embodiments, the valve is made of a single material; this improves the control of quality, ease of manufacture, and cost of fabrication. Preferably, the valve is made primarily of a synthetic material. More preferably, the material used is also flexible, durable, and commercially available or easy to make. Suitable materials for use in creating the valve of the present disclosure include, but are not limited to, polyurethanes, polyesters, polyethylenes, hydrogels, collagen, elastin, and silicone. One preferred material for use comes from the hydrogel group: poly(vinyl alcohol) cryogel (PVA cryogel) (Ku et al., U.S. Pat. No. 5,981,826). PVA cryogel is a hydrogel that has been shown to have low thrombogenicity (Miyake H, Handa H, Yonekawa Y, Taki W, Naruo Y, Yamagata S, Ikada Y, Iwata H, Suzuki M, New Sinall-Caliber Antithrombotic Vascular Prosthesis: Experimental Study, Microsurgery. 1984; 5(3):144-50).

PVA cryogel can be manufactured as described in U.S. Pat. No. 5,981,826, which is hereby incorporated by reference herein. In using any of the aforementioned prescribed materials, molding is the preferred method to fabricate the valve of the present disclosure, and can be conducted by those familiar with the general art of molding.

In some preferred embodiments, the central portion of the generally cylindrical tube has a lower compliance relative to the flared inlet, flared outlet and the leaflets. One method of lowering the valve tube compliance involves adding a second material to the material used to make the tube (e.g. PVA cryogel). The added material is in a form capable of strengthening/stiffening the primary material such as, but not limited to, particulate forms such as, but not limited to, filaments, strands, or thin rods, thus creating a composite tube. The additional material preferably has a Young's Modulus greater than that of the primary material in the valve, and should preferably be biocompatible, and have relatively low thrombogenicity. Exemplary stiffening materials include, but are not limited to, PET, ePTFE, nitinol, and cobalt-chromium alloys. The stiffening material could also be a stent integrated into the tube material to not only stiffen the tube but also to act as an implantation device. In a preferred embodiment, the Young's modulus of the tube portion of the valve is about 50 kilo-Pascals to about 100 giga-Pascals, and the leaflets have a Young's modulus of about 50 kilo-Pascals to about 5 giga-Pascals.

Preferably, the valve contains a radiopaque marker to facilitate delivery, orientation, and placement of the valve using intravenous catheter approaches. Such markers are preferably biocompatible, have low thrombogenicity, and are preferably cast into the valve inlet and outlet. Radiopaque marker(s) can be added to the valve via methods commonly known to those familiar with the art of manufacturing medical devices. Exemplary radiopaque markers suitable for use with a valve according to the present invention include, but are not limited to, platinum, iridium, and nickel titanium alloys.

The descriptions above detailing certain exemplary embodiments contain specificities and are intended only to best illustrate the design and function of the valve of the present disclosure for a person of ordinary skill in the art to become knowledgeable and enabled to utilize the present disclosure for its appropriate purposes. The descriptions are neither exhaustive nor meant to limit the scope of the present disclosure to the specificities disclosed above. Many variations and modifications may be made to the above-described embodiments of the present disclosure without departing substantially from the spirit and principles of the present disclosure. All such modifications and variations are intended to be included herein within the scope of this disclosure and protected by the following claims.

Having generally described prosthetic valves according to the present disclosure and methods of making and using such valves, the examples that follow describe some specific embodiments. While embodiments of the valves and methods of making and using the valves are described in connection with the following examples and the corresponding text, there is no intent to limit embodiments to these examples. On the contrary, the intent is to cover all alternatives, modifications, and equivalents included within the scope of the disclosure.

### EXAMPLES

Five valves of the same prototype design were fabricated in accordance with preferred embodiments of the present disclosure and were tested for static and dynamic pressure performance to verify their functionality. The valves were tested to assess each valve's performance based on three design criteria: 1) that the valve open with a distal pressure gradient less than 5.0 mmHg, 2) that the valve withstand 300 mmHg of backpressure with leakage less than 1.0 mL/min, and 3) that the valve meet criteria 1 and 2 after at least 500,000 cycles of operation.

The information contained herein this Example section is provided to illustrate the utility of the present disclosure via one particular embodiment. Multiple embodiments and variations may exist, and the features of the valves of the present disclosure are not limited to the embodiment presented in the present Examples.

Valve Fabrication: Each of the five valves was created from the same mold such that dimensional differences were negligible. The valves were made of 15 percent poly(vinyl alcohol) (PVA) cryogel solution, manufactured per the guidelines of U.S. Pat. No. 5,981,826 (Ku, et al., which is hereby incorporated by reference herein in its entirety). The PVA cryogel was injected into a two-part cavity mold made of silicone rubber to create the valve shape, and the cryogel was cured using alternative cycles of freezing and thawing, per the guidelines of U.S. Pat. No. 5,981,826 (incorporated by reference above). The cavity molds were created by forming them around four positive valve dies using standard molding techniques. Each of the positive dies were fabricated using stereolithographic techniques. These techniques are known to those familiar with the art of mold-making. With each die having the same dimensions, four identical valves could be fabricated per batch in one silicone mold.

The outer diameter of the central portion of the valve tube and the inner diameter of the test “vein”, described below, (D) were the same, at 10.0 mm. The outer diameter of the flared inlet and outlet was approximately 11.7 mm. The thickness of the valve tube wall was about 1.3 mm, or 0.13 D. The total length of the valve was approximately 20 mm, or 2 D. The thickness of the leaflets was about 0.9 mm, or 0.09 D. Angle α and angle β were both approximately 30 degrees, angle χ was approximately 60 degrees, and angle θ was approximately 150 degrees. Variable radius fillets were used to smooth angled joints on the flared inlet and outlet and the leaflets, and the radii on various fillets ranged from 0.2 mm to 5 mm.

Opening Pressure Testing: It is important for the valve to have a low opening pressure to ensure that blood continues to flow forward to the heart. Typically, the pressure that opens natural vein valves is less than 5.0 mmHg, and thus a prosthetic valve will optimally perform similarly. All five prototype valves were tested as described below.

Each valve was affixed in a 10 mm inner diameter (D) viscoelastic tube (the test “vein”), serving as a model of implantation in a human vein. The tube's elastic behavior is similar to that of human veins at low pressures. The vein-like tube was connected to a hand-syringe pump and an in-line pressure transducer to measure pressure forces. The entire flow set-up was placed in a horizontal position to negate the effects of gravitational forces. The valves were orientated such that the proximal end was exposed to ambient pressure. Distal pressure was applied via a syringe pump in 1 mmHg increments. The proximal end of the valve was visually monitored for the passing of water. Upon appearance of water, the opening pressure was recorded.

Five trials were conducted for each valve, with approximately one minute elapsing between each trial. Opening pressure ranged between 2.3±0.7 mmHg and 3.7±0.7 mmHg. These opening pressures indicate that the valve of the present disclosure is well suited for human venous anatomy of the lower extremities, which typically experience opening pressures around 4 mmHg. This testing shows that the valve of the present disclosure will allow blood to flow easily towards the heart.

Backpressure Testing: All five prototype valves were subjected to backpressure testing. Each valve was orientated in the experimental setup such that its distal end was exposed to ambient pressure. Static pressure was applied at 20 mmHg increments, from 0 to 300 mmHg. Pressure was applied via the syringe pump for a of 30 second duration, with a 5 second duration for ramping the pressure to the desired level. Leak rate was measured on a basis of volume of leakage per minute, a standard venous metric. Leak rates were measured at each sustained pressure level, from 0 to 300 mmHg, in 20 mmHg increments.

All five valves met the design criteria for leakage under high backpressure. Four of the five valves allowed less than 0.5 mL/min leakage at 300 mmHg, and one valve allowed less than 1.0 mL/min of leakage at 300 mmHg. Pressure in the common femoral vein, a deep vein that is an ideal region for implantation, is typically between 60 and 100 mmHg. Thus, the valve of the present disclosure is very well suited for preventing reflux in the deep veins due to high backpressure.

Cyclic Life Testing: This experimental setup was used to evaluate the valve's robustness and lifespan. One prototype valve was placed in a cyclic flow loop, designed to open and close the valve numerous times in simulated physiologic conditions. Physiologically, native vein valves open and close synchronously with calf muscle contractions. During normal walking, calf compression propels 10 to 20 mL of blood through the veins, and compression occurs about 40 times per minute (0.67 Hz) during normal cadence. The primary requirement during cyclic testing was that the valve opened and closed in a periodic, identifiable fashion. Correspondingly, a rotary phase pump was used to displace a total of approximately 10 mL of water with each stroke through the valve orifice, at a frequency of 0.70±0.03 Hz. These parameters are consistent with the frequency and volume of blood flow during calf compression in normal walking cadence. The total net flow of water was 450±30 mL/min.

The flow loop was designed so that hydrostatic pressure was maintained proximal to the valve at 50±5 mmHg, similar to physiologic conditions proximal to a typical adult femoral vein. The valve was orientated such that the proximal end was facing upwards, exposed to the pressure of the water column above it, thus mimicking a leg vein valve. The valve was evaluated for functional performance at various points during the cyclic testing, typically every 50,000 to 60,000 cycles (about 24 hours). At each measurement interval, the test specimen was removed from the loop and exposed to opening pressure and backpressure testing. This testing quantified the valve's functionality with respect to reflux leak-rate performance and opening pressure characteristics at various stages of life cyclic testing. FIG. 8 below depicts the opening pressure performance of the valve, while FIG. 9 depicts the minimal leakage allowed by the valve. The valve withstood over 500,000 cycles of operation in opening and closing, and remained fully functional with respect to the critical design criteria. Testing was stopped after 500,000 cycles because test equipment began to deteriorate; valve performance, however, remained functional.

Burst Pressure Testing: Three of the prototype valves were subjected to burst pressure testing. Burst pressure results ranged from 530±10 mmHg to 940±10 mmHg. The minimum burst pressure of 530±10 mmHg is six times greater that normal physiologic conditions, indicating that the valve will be sufficiently robust to withstand the proximal pressure of leg veins.

It should be emphasized that the above-described embodiments are merely possible examples of implementations. Many variations and modifications may be made to the above-described embodiments. All such modifications and variations are intended to be included herein within the scope of this disclosure and protected by the following claims.

What is claimed is:

1. A prosthetic valve for implantation in a vessel, wherein the valve allows primarily one-way flow of a fluid within the vessel and wherein the valve comprises a generally cylindrical tube comprising:
an outer surface defining an outer diameter,
an inner surface defining an inner diameter,
an inlet,
an outlet,
a central portion between the inlet and the outlet, and
at least two leaflets, wherein, when the valve is in a relaxed state, the leaflets are at least partially in contact with one another and substantially prevent a fluid from flowing from the outlet to the inlet, and wherein, upon application of pressure from the direction of the inlet, the leaflets separate from contact to allow a fluid to flow from the inlet to the outlet.

2. The valve of claim 1, wherein the inlet is flared.

3. The valve of claim 1, wherein the outlet is flared.

4. The valve of claim 1, wherein the inlet and the outlet are flared.

5. The valve of claim 2, wherein the flared inlet forms an angle γ with the outer surface of the central portion of the generally cylindrical tube, wherein the angle γ is from about 90 to about 270 degrees.

6. The valve of claim 5, wherein the joint formed by the meeting of the flared inlet and the central portion of the generally cylindrical tube is smoothed in a manner such that it can be defined by a parametric surface having curvatures of various radii.

7. The valve of claim 6, wherein the curvatures of the parametric surface have radii ranging from about 0 R to about 10 R, wherein R is the radius of the inner surface of the central portion of the generally cylindrical tube.

8. The valve of claim 3, wherein the flared outlet forms an angle δ with the outer surface of the central portion of the generally cylindrical tube, wherein the angle δ is from about 90 to about 270 degrees.

9. The valve of claim 8, wherein the joint formed by the meeting of the flared outlet and the central portion of the generally cylindrical tube is smoothed in a manner such that it can be defined by a parametric surface having curvatures of various radii.

10. The valve of claim 9, wherein the curvatures of the parametric surface have radii ranging from about 0 R to about 10 R, wherein R is the radius of the inner surface of the central portion of the generally cylindrical tube.

11. The valve of claim 1, wherein the outer diameter of the central portion of the tube is about 0.75 D to about 1.50 D, wherein D is the un-collapsed inner diameter of the vein at low pressures.

12. The valve of claim 1, wherein the outer diameter of the central portion of the tube is about 1 millimeter to about 50 millimeters.

13. The valve of claim 1, wherein the valve has a length of about 0.5 D to about 4 D, wherein D is the un-collapsed inner diameter of the vein at low pressures.

14. The valve of claim 1, wherein the valve has a length of about 2 millimeters to about 50 millimeters.

15. The valve of claim 1, wherein the inlet and outlet preferably each have a length of about 0.2 D to about 1 D, wherein D is the un-collapsed inner diameter of the vein at low pressures.

16. The valve of claim 1, wherein the generally cylindrical tube further comprises a wall, defined in part by the inner surface and outer surface, wherein the wall has a thickness of about 0.05 D to about 0.15 D, wherein D is the un-collapsed inner diameter of the vein at low pressures.

17. The valve of claim 1, wherein the leaflets have a thickness of about 0.01 D to about 0.2 D, wherein D is the un-collapsed inner diameter of the vein at low pressures.

18. The valve of claim 1, wherein the valve comprises two leaflets, and wherein each leaflet comprises:
a) a distal half approximating half of an elliptical plate and oriented substantially parallel to an angled transverse plane passing through the cylindrical tube, forming an angle χ with the outer surface of the central portion of the generally cylindrical tube which is from about 30 degrees to about 60 degrees, and
b) a proximal half approximating a trapezoidal plate and generally aligned substantially parallel to a plane containing the longitudinal axis of the tube,
wherein the distal and proximal halves of each leaflet connect to form a joint having an angle θ, wherein the joint is smoothed in a manner such that the joint can be defined by a parametric surface having curvatures of various radii.

19. The valve of claim 18, wherein angle θ is between about 100 degrees and about 170 degrees.

20. The valve of claim 18, wherein the curvatures of the parametric surface have radii ranging from about 0.5 D and about 5 D, wherein D is the un-collapsed inner diameter of the vein at low pressures.

21. The valve of claim 1, wherein a joint formed between each leaflet and the inner surface of the tube is flexible and is smoothed in a manner such that it can be defined by a parametric surface having curvatures of various radii.

22. The valve of claim 21, wherein the curvatures of the parametric surface have radii ranging from about 0.5 D and about 10 D, wherein D is the un-collapsed inner diameter of the vein at low pressures

23. The valve of claim 1, wherein the outer surface of the generally cylindrical tube further comprises a material to facilitate intimal growth and healing of the vessel containing the implanted valve.

24. The valve of claim 23, wherein the material is a biocompatible material selected from: a mesh, a net, an arrangement of filaments, an arrangement of fibers, and a combination thereof.

25. The valve of claim 1, wherein at least a portion of the valve is made of a composite material.

26. The valve of claim 25, wherein the composite material comprises a volume fraction of about 0 to about 50 percent of a particulate material.

27. The valve of claim 26, wherein the particulate material is selected from: fibers, filaments, strands, grains, and a combination thereof.

28. The valve of claim 1, wherein the valve is non-thrombogenic.

29. The valve of claim 1, wherein the valve further comprises one or more anti-thrombogenic agents coated thereon or incorporated therein, or both.

30. The valve of claim 20, wherein the anti-thrombogenic agent comprises an agent selected from: heparin, warfarin sodium, sulfated polysaccharides, prostaglandins, and albumin.

31. The valve of claim 1, wherein the valve comprises a synthetic material.

32. The valve of claim 31, wherein the synthetic material comprises a material selected from: polyurethanes, polyesters, polyethylenes, hydrogels, collagen, elastin, and silicone.

33. The valve of claim 31, wherein the material is poly(vinyl alcohol) cryogel (PVA cryogel).

34. The valve of claim 1, wherein the valve is biocompatible.

35. The valve of claim 4, wherein the flared inlet and flared outlet have greater compliance than the central portion of the valve and the leaflets.

36. The valve of claim 1, wherein the central portion of the tube has a lower compliance than the remainder of the valve.

37. The valve of claim 1, wherein the central portion tube has a Young's modulus of about 50 kilo-Pascals to about 100 giga-Pascals.

38. The valve of claim 1, wherein the leaflets have greater compliance than the remainder of the valve.

39. The valve of claim 1, wherein the leaflets comprise a material having a Young's modulus of about 50 kilo-Pascals to about 5 giga-Pascals.

40. The valve of claim 1, further comprising a radiopaque material.

41. The valve of claim 40, wherein the radiopaque material comprises a material selected from: platinum, iridium, and nickel titanium alloys.

42. The valve of claim 4, wherein the flared inlet and flared outlet can independently elastically expand in the radial direction and can increase in radius by a value of about 0 R to about 1.0 R, where R is an inner radius of the central portion of the generally cylindrical tube.

43. The valve of claim 1, wherein the central portion of the generally cylindrical tube can elastically expand in the radial direction and can increase in radius by a value of about 0 R to about 0.5 R, where R is an inner radius of the central portion of the generally cylindrical tube.

44. The valve of claim 1, wherein the valve can elastically expand in the axial direction and can increase its total length by a value of about 0L to about 0.5L, where L is the total length of the valve in the axial direction.

45. A prosthetic valve for implantation in a vessel, wherein the valve allows primarily one-way flow of a fluid within the vessel and wherein the valve comprises a generally cylindrical tube comprising:
an outer surface defining an outer diameter,
an inner surface defining an inner diameter,
a flared inlet,
a flared outlet,
a central portion between the inlet and the outlet, and
at least two leaflets, wherein, when the valve is in a relaxed state, the leaflets are at least partially in contact with one another and substantially prevent a fluid from flowing from the outlet to the inlet, and wherein, upon application of pressure from the direction of the inlet, the leaflets separate from contact to allow a fluid to flow from the inlet to the outlet.

46. A method of implanting the valve of claim 1 into the vessel of a patient comprising:
delivering the valve to an implantation site within the vessel,
placing the valve in a proper orientation, and
securing the valve in place within the vessel.

47. The method of claim 46 wherein delivering the valve to the implantation site comprises delivery via an intravenous catheter.

48. The method of claim 46, wherein delivering the valve to the implantation site comprises delivery via a venotomy.

49. The method of claim 46, wherein securing the valve in place within the vessel comprises the use of one or more endovascular implantation techniques selected from: sutures, a balloon-expandable stent, a self-expanding stent, hooks, and barbs.
